SEQUENCE LISTING (8 sequences)

SEQ ID NO: 1 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 112 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKEGYQDYEPEA

SEQ ID NO: 3 - protein, 126 aa, Homo sapiens
MDVFMKGLSKAKEGVVAAAEKTKQGVAEAAGKTKEGVLYVVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 4 - protein, 159 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKSSLRNECSPEPAASTDQSQNEEGAPQEGILEDMPVDPDNEAYEMPSEEGYQDYEPEA

SEQ ID NO: 5 - protein, 151 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEPPCCEEAQATWRGHAMCTCSS

SEQ ID NO: 6 - protein, 115 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKVWLCTFCVTFISW

SEQ ID NO: 7 - protein, 66 aa, Homo sapiens
MDVFMKGLSKAKEGVVAAAEKTKQGVAEAAGKTKEGVLYVGSKTKEGVVHGVATVAEKTKEQVTNV

SEQ ID NO: 8 - protein, 98 aa, Homo sapiens
MDVFMKGLSKAKEGVVAAAEKTKQGVAEAAGKTKEGVLYVVAEKTKEQVTNVGGAVVTGVTAVAQKTVEGAGSIAAATGFVKKDQLGKEGYQDYEPEA

(19) United States
(12) Patent Application Publication
(10) Pub. No.: US 2025/0012814 A1
(43) Pub. Date: Jan. 9, 2025

(54) METHODS OF DIAGNOSING A SYNUCLEINOPATHY

(71) Applicant: Nitrase Therapeutics, Inc., Brisbane, CA (US)

(72) Inventors: Sami HUSSAIN, San Francisco, CA (US); Irene GRISWOLD-PRENNER, Jackson, WY (US)

(21) Appl. No.: 18/706,424

(22) PCT Filed: Oct. 31, 2022
(86) PCT No.: PCT/US2022/078980

§ 371 Date: May 1, 2024

Related U.S. Application Data
(60) Provisional application No. 63/274,237, filed on Nov. 1, 2021.

(51) Int. Cl. G01N 33/68

(52) U.S. Cl. CPC G01N 33/6896; G01N 2333/4703; G01N 2800/2814; G01N 2800/2835

(57) ABSTRACT

A method for diagnosis and the treatment of a synucleinopathy related to nitration of alpha-synuclein, the method comprising: determining the expression level of at least one nitrated alpha-synuclein protein selected from the group consisting of an alpha-synuclein protein nitrated at tyrosine residue 39 nY39, nY125, nY133, and nY136 in an alpha-synuclein protein in a cerebrospinal fluid (CSF) sample obtained from the subject: and comparing the expression level of the at least one nitrated alpha-synuclein protein to a reference level of the at least one nitrated alpha-synuclein protein, wherein an increase in the expression level of the at least one nitrated alpha-synuclein protein in the CSF sample relative to the reference expression level of the at least one nitrated alpha-synuclein protein identifies a subject having a synucleinopathy.

### SEQUENCE LISTING

The instant application contains a Sequence Listing which has been submitted electronically in XML format and is hereby incorporated by reference in its entirety. Said XML copy, created on Oct. 26, 2022, is named “51340-010WO2_Sequence_Listing_10_26_22” and is 8,658 bytes in size.

### BACKGROUND OF THE INVENTION

Nitration of proteins has been linked to various disease conditions through inducement of agglomeration of proteins. Nitration is known to play a role in neurodegenerative diseases, e.g., synucleinopathies e.g., Parkinson's disease (PD), Alzheimer's disease (AD), amyotrophic lateral sclerosis (ALS), and Huntington's disease. For example, the relevance of nitration has been demonstrated for alpha-synuclein in aging and diseases.

For example, Parkinson's disease is the second most common neurodegenerative disease, affecting one million people in the United States alone. The disease is characterized by a dramatic loss of dopaminergic neurons in the substantia nigra, with up to 80% of these neurons having degenerated by the time of clinical manifestations. Death of these neurons causes tremor, bradykinesia, rigidity, and postural instability. Currently, there is no effective intervention to reverse disease progression, suggesting the need for early biomarkers and prophylactic treatments. Based on the loss of dopamine or dopaminergic dysfunction in PD patients, most of the current therapies mainly rely on the use of dopaminergic pharmacological agents, such as levodopa (L-DOPA), for the treatment of the clinical symptoms of PD. However, as L-DOPA is quickly metabolized, it often leads to poor efficacy and severe peripheral side effects. In particular, while L-DOPA may be effective for symptomatic relief, its efficacy is diminished and it causes medication-related complications, particularly motor fluctuations and dyskinesia, such as L-DOPA-induced dyskinesia, after chronic or long-term use. In accordance with these severe contraindications, including drug-related problems due to inappropriate L-DOPA timing of administration, PD is a challenging pathology to treat and patients are advised to take their medications on time, every time. Despite the intensive efforts in PD research and development, the need for a state biomarker of PD is obvious, and there are clear unmet medical needs for the development of treatment options to slow the trajectory of emergence of PD and/or strengthen treatment options to enhance patients' quality of life.

A hallmark of PD pathology is an intracellular accumulation of alpha-synuclein aggregates resulting in Lewy Bodies and Lewy Neurites. It has been hypothesized that these alpha-synuclein aggregates, in some form, are responsible for causing PD. Additionally, the presence of post-translational modifications (PTMs) to the alpha-synuclein protein have been correlated to pathology formation. Among the most commonly observed alpha-synuclein PTMS in human PD brains are nitration at tyrosine (Y) residues (e.g., Y39, Y125, Y133, and Y136), N-terminal acetylation, phosphorylation, ubiquitination, and N- and C-terminal truncations. In particular, phosphorylation at serine 129 has been extensively studied in biological fluids (e.g., cerebrospinal fluid (CSF), plasma, and saliva) and nitrated Y39 (nY39) has been detected in the blood of patients with PD. However, the lack of antibodies that specifically bind these PTMs have hampered further studies aimed at elucidating their role in PD. Accordingly, the distribution of alpha-synuclein PTMs, particularly in the CSF, remain poorly understood.

The present disclosure relates to useful methods and compositions for the diagnosis and/or treatment of a synucleinopathy (e.g., PD, multiple system atrophy, and dementia with Lewy Bodies) related to the identification of nitrated alpha-synuclein, such as nY39 and/or nY136, in the CSF of subjects as a biomarker of a synucleinopathy.

### SUMMARY

Alpha-synuclein has been shown to be nitrated in the inclusions present in Parkinson's disease (PD), dementia with Lewy Bodies (DLB), Lewy body variant of AD, and Multiple-System Atrophy (MSA). Several studies have suggested that the insolubility of alpha-synuclein may be a result of nitration since nitrated alpha-synuclein is present in the insoluble (not soluble) fraction of homogenates from PD brains. Alpha-synuclein nitration also induces oligomerization and β-pleated (beta-pleated) sheet formation in a nucleation-dependent manner. Furthermore, nitration of alpha-synuclein results in its removal from lipid vesicles and increases its half-life due to decreased ability of the proteasome to degrade it. Each of these factors contributes to alpha-synuclein accumulation and aggregation.

A role for the adverse effects of alpha-synuclein nitration and dityrosine crosslinking was also shown in two independent in vivo models of PD. One model mimics alpha-synuclein dityrosine crosslinking in an alpha-synuclein transgenic mouse model in which Y39 is replaced with cysteine facilitating inter-synuclein disulfide crosslinking. In a second model, nitrated alpha-synuclein was microinjected into dopaminergic neurons in the rat ventral mesencephalon. In both in vivo PD models, alpha-synuclein aggregation and either tyrosine hydroxylase positive cell loss and motor deficits or cognitive and motoric dysfunction were observed. The data suggest a role of alpha-synuclein nitration in mediating disease pathology and dysfunction in synucleinopathies, such as PD, DLB, and MSA. Finding a diagnostic method related to this nitration event could be highly advantageous for the treatment of synucleinopathies, such as PD, DLB, and MSA and symptoms thereof.

This invention is based, at least in part, on the discovery that levels of nitrated alpha-synuclein protein in the cerebrospinal fluid (CSF), can be used to diagnose and identify patients having a synucleinopathy (e.g., DLB, MSA, PD, or a subtype of the disease thereof e.g., prodromal PD). Accordingly, the disclosure provides methods of diagnosing and treating, subjects (e.g., human patients) based on this discovery. The methods of the invention can also be used to obtain a differential diagnosis of one synucleinopathy over another (e.g., PD vs AD).

In an aspect, the invention features a method of diagnosing a synucleinopathy in a subject, the method including: (a) determining the expression level of at least one nitrated alpha-synuclein protein selected (e.g., 1, 2, 3, or 4 nitrations per alpha-synuclein protein (e.g., nY39/nY136) and/or 1, 2, 3, or 4 nitrated alpha-synuclein proteins (e.g., alpha-synuclein nY36 and alpha-synuclein nY136)) from the group consisting of an alpha-synuclein protein nitrated at tyrosine residue 39 (nY39), nY125, nY133, and nY136 in an alpha-synuclein protein in a CSF sample obtained from the subject; and (b) comparing the expression level of the at least one nitrated alpha-synuclein protein to a reference level of the at least one nitrated alpha-synuclein protein, wherein an increase in the expression level of the at least one nitrated alpha-synuclein protein in the CSF sample relative to the reference expression level of the at least one nitrated alpha-synuclein protein identifies a subject having a synucleinopathy.

In some embodiments, the expression level of at least two nitrated alpha-synuclein proteins selected from the group consisting of nY39, nY125, nY133, and nY136 in a CSF sample obtained from the subject are determined. For example, in some embodiments, the at least two nitrated alpha-synuclein proteins are nY39 and nY136. In some embodiments, the nitrated alpha-synuclein is nY39. In some embodiments, the nitrated alpha-synuclein is nY136. In some embodiments, the one nitrated alpha-synuclein protein is nY39 and nY136.

In some embodiments, a statistically significant increase in the expression level of the at least one nitrated alpha-synuclein protein in the CSF sample relative to the reference expression level of the at least one nitrated alpha-synuclein protein identifies a subject having a synucleinopathy.

In some embodiments, (a) the expression level of at least two nitrated alpha-synuclein proteins is an average of the at least two (e.g., at least three, at least four, or at least five) nitrated alpha-synuclein protein of the CSF sample; (b) the reference expression level of the at least two nitrated alpha-synuclein proteins are an average of the at least two nitrated alpha-synuclein proteins of the reference; and (c) the average of the at least two nitrated alpha-synuclein proteins of the CSF sample are compared to the average of the at least two nitrated alpha-synuclein proteins of the reference.

In some embodiments, the method further includes administering to the subject an effective amount of a cognition-enhancing agent (e.g., donepezil, rivastigmine tartrate, galantamine HBr, memantine, and modafinil), an antidepressant agent (e.g., sertraline, fluoxetine, citalopram, escitalopram, paroxetine, and fluvoxamine), a dopamine promoter (e.g., agonist e.g., selegiline, pramipexole and levodopa (L-DOPA)), an anti-tremor agent (e.g., propranolol, primidone, gabapentin, and topiramate), and/or a neuroprotective agent (e.g., a gene encoding all or a portion of Kifap3, Bcl-xl, Crmp1, Chk.beta., CALM2, Caly, NPG11, NPT1, Eef1a1, Dhps, Cd151, Morf412, CTGF, LDH-A, Atl1, NPT2, Ehd3, Cox5b, Tuba1a, gamma-actin, Rpsa, NPG3, NPG4, NPG5, NPG6, NPG7, NPG8, NPG9, or NPG1; gangliosides, topiramate, riluzole, methylprednisolone, rivstigmine, selegiline, cilostazol, rasagiline, tenocyclidine, 7-nitroindazole, N-(3-propylcarbamoyloxirane-2-carbonyl)-isoleucyl-proline, huperzine A, SGS-742, D-JNKI-1, nalmefene, ziconotide, dexanabinol, remacemide, clomethiazole, propentofylline, Z-Val-Ala-Asp fluoromethyl ketone, piracetam, epigallocatechin gallate, vinpocetine, tempol, butylphthalide, eliprodil, tirilazad, nefiracetam, gacyclidine, nizofenone, meclofenoxate, linopiridine, fosfructose, methylprednisolone hemisuccinate, dextrorphan, ebselen, almitrine, brimapitide, edaravone, edaravone, minocycline, epoetin-β, trafermin, filgrastim, eicosapentaenoic acid, and/or pioglitazone).

In some embodiments, the reference expression level is the mean level of expression of the at least one nitrated alpha-synuclein protein in a population of subjects.

In some embodiments, the expression level is a protein expression level. In some embodiments, the protein expression level is determined by single molecule detection, western blot, enzyme-linked immunoassay (ELISA), immunohistochemistry, or mass spectrometry.

In another aspect, the invention features a method of identifying a subject having a synucleinopathy, the method including determining the protein level of alpha-synuclein including a nitration of Y39, Y125, Y133, and/or Y136 in an alpha-synuclein protein in a CSF sample obtained from the subject, wherein the presence of nY39, nY125, nY133, and/or nY136 identifies the subject as having a synucleinopathy.

In another aspect, the invention features a method of diagnosing a synucleinopathy in a subject, the method including determining the protein level of alpha-synuclein including a nitration of Y39, Y125, Y133, and/or Y136 in an alpha-synuclein protein in a CSF sample obtained from the subject, wherein the presence of nY39, nY125, nY133, and/or nY136 identifies the subject as having a synucleinopathy.

In some embodiments of any of the foregoing aspects, the protein expression level is determined by single molecule detection, western blot, ELISA, immunohistochemistry, or mass spectrometry.

In some embodiments, the presence of nY39 identifies the subject as having a synucleinopathy. In some embodiments, the presence of nY136 identifies the subject as having a synucleinopathy. In some embodiments, the presence of nY39 and nY136 identifies the subject as having a synucleinopathy.

In particular embodiments of any of the above methods, the method includes the step of assessing whether the subject has PD or which synucleinopathy the subject has (e.g., PD or AD).

In some embodiments, the method further includes administering to the subject a therapeutically effect amount of a cognition-enhancing agent (e.g., donepezil, rivastigmine tartrate, galantamine HBr, memantine, and modafinil), an antidepressant agent (e.g., sertraline, fluoxetine, citalopram, escitalopram, paroxetine, and fluvoxamine), a dopamine promoter (e.g., agonist e.g., selegiline, pramipexole and levodopa (L-DOPA)), an anti-tremor agent (e.g., propranolol, primidone, gabapentin, and topiramate), and/or a neuroprotective agent (e.g., a gene encoding all or a portion of Kifap3, Bcl-xl, Crmp1, Chk.beta., CALM2, Caly, NPG11, NPT1, Eef1a1, Dhps, Cd151, Morf412, CTGF, LDH-A, Atl1, NPT2, Ehd3, Cox5b, Tuba1a, gamma-actin, Rpsa, NPG3, NPG4, NPG5, NPG6, NPG7, NPG8, NPG9, or NPG1; gangliosides, topiramate, riluzole, methylprednisolone, rivstigmine, selegiline, cilostazol, rasagiline, tenocyclidine, 7-nitroindazole, N-(3-propylcarbamoyloxirane-2-carbonyl)-isoleucyl-proline, huperzine A, SGS-742, D-JNKI-1, nalmefene, ziconotide, dexanabinol, remacemide, clomethiazole, propentofylline, Z-Val-Ala-Asp fluoromethyl ketone, piracetam, epigallocatechin gallate, vinpocetine, tempol, butylphthalide, eliprodil, tirilazad, nefiracetam, gacyclidine, nizofenone, meclofenoxate, linopiridine, fosfructose, methylprednisolone hemisuccinate, dextrorphan, ebselen, almitrine, brimapitide, edaravone, edaravone, minocycline, epoetin-β, trafermin, filgrastim, eicosapentaenoic acid, and/or pioglitazone).

In another aspect, the invention features a method of treating a subject having a synucleinopathy, the method including administering to the subject a therapeutically effective amount of a cognition-enhancing agent (e.g., donepezil, rivastigmine tartrate, galantamine HBr, memantine, and modafinil), an antidepressant agent (e.g., sertraline, fluoxetine, citalopram, escitalopram, paroxetine, and fluvoxamine), a dopamine promoter (e.g., agonist e.g., selegiline, pramipexole and levodopa (L-DOPA)), an anti-tremor agent (e.g., propranolol, primidone, gabapentin, and topiramate), and/or a neuroprotective agent (e.g., a gene encoding all or a portion of Kifap3, Bcl-xl, Crmp1, Chk.beta., CALM2, Caly, NPG11, NPT1, Eef1a1, Dhps, Cd151, Morf412, CTGF, LDH-A, Atl1, NPT2, Ehd3, Cox5b, Tubala, gamma-actin, Rpsa, NPG3, NPG4, NPG5, NPG6, NPG7, NPG8, NPG9, or NPG1; gangliosides, topiramate, riluzole, methylprednisolone, rivstigmine, selegiline, cilostazol, rasagiline, tenocyclidine, 7-nitroindazole, N-(3-propylcarbamoyloxirane-2-carbonyl)-isoleucyl-proline, huperzine A, SGS-742, D-JNKI-1, nalmefene, ziconotide, dexanabinol, remacemide, clomethiazole, propentofylline, Z-Val-Ala-Asp fluoromethyl ketone, piracetam, epigallocatechin gallate, vinpocetine, tempol, butylphthalide, eliprodil, tirilazad, nefiracetam, gacyclidine, nizofenone, meclofenoxate, linopiridine, fosfructose, methylprednisolone hemisuccinate, dextrorphan, ebselen, almitrine, brimapitide, edaravone, edaravone, minocycline, epoetin-β, trafermin, filgrastim, eicosapentaenoic acid, and/or pioglitazone), wherein the expression level of a nitrated alpha-synuclein protein having at least one of the following tyrosine residues Y39, Y125, Y133, and Y136 in an alpha-synuclein protein nitrated in a CSF sample obtained from the subject has been determined to be increased relative to a reference expression level of the at least one nitrated alpha-synuclein protein e.g., 1, 2, 3, or 4 nitrations per alpha-synuclein protein (e.g., nY39/nY136)).

In some embodiments, a nitrated alpha-synuclein protein having at least two of the following tyrosine residues Y39, Y125, Y133, and Y136 in an alpha-synuclein protein nitrated in a CSF sample obtained from the subject has been determined.

In some embodiments, the expression level of a nitrated alpha-synuclein protein having the tyrosine residue Y39 nitrated in a CSF sample obtained from the subject has been determined. In some embodiments, the expression level of a nitrated alpha-synuclein protein having the tyrosine residue Y136 nitrated in a CSF sample obtained from the subject has been determined. In some embodiments, the expression level of a nitrated alpha-synuclein protein having the tyrosine residues Y39 and Y136 nitrated in a CSF sample obtained from the subject have been determined.

In another aspect, the invention features a method of treating a subject having a synucleinopathy, the method including: (a) determining the protein expression level of a nitrated alpha-synuclein protein including a nitration of Y39, Y125, Y133, and/or Y136 in an alpha-synuclein protein in a CSF sample obtained from the subject, wherein an increase in the expression level of the nitrated alpha-synuclein protein relative to a reference expression level identifies the subject as having a synucleinopathy; and (b) administering to the subject a therapeutically effective amount of a cognition-enhancing agent (e.g., donepezil, rivastigmine tartrate, galantamine HBr, memantine, and modafinil), an antidepressant agent (e.g., sertraline, fluoxetine, citalopram, escitalopram, paroxetine, and fluvoxamine), a dopamine promoter (e.g., agonist e.g., selegiline, pramipexole and levodopa (L-DOPA)), an anti-tremor agent (e.g., propranolol, primidone, gabapentin, and topiramate), and/or a neuroprotective agent (e.g., a gene encoding all or a portion of Kifap3, Bcl-xl, Crmp1, Chk.beta., CALM2, Caly, NPG11, NPT1, Eef1a1, Dhps, Cd151, Morf412, CTGF, LDH-A, Atl1, NPT2, Ehd3, Cox5b, Tuba1a, gamma-actin, Rpsa, NPG3, NPG4, NPG5, NPG6, NPG7, NPG8, NPG9, or NPG1; gangliosides, topiramate, riluzole, methylprednisolone, rivstigmine, selegiline, cilostazol, rasagiline, tenocyclidine, 7-nitroindazole, N-(3-propylcarbamoyloxirane-2-carbonyl)-isoleucyl-proline, huperzine A, SGS-742, D-JNKI-1, nalmefene, ziconotide, dexanabinol, remacemide, clomethiazole, propentofylline, Z-Val-Ala-Asp fluoromethyl ketone, piracetam, epigallocatechin gallate, vinpocetine, tempol, butylphthalide, eliprodil, tirilazad, nefiracetam, gacyclidine, nizofenone, meclofenoxate, linopiridine, fosfructose, methylprednisolone hemisuccinate, dextrorphan, ebselen, almitrine, brimapitide, edaravone, edaravone, minocycline, epoetin-β, trafermin, filgrastim, eicosapentaenoic acid, and/or pioglitazone).

In some embodiments, the nitrated alpha-synuclein protein includes a nitration of Y39. In some embodiments, the nitrated alpha-synuclein protein includes a nitration of Y136. In some embodiments, the nitrated alpha-synuclein protein includes a nitration of Y39 and Y136.

In some embodiments of any of the foregoing aspects, an increase is a statistically significant increase, wherein the statistically significant increase is an increase is at least a 10%, 25%, 50%, 100%, 2-fold, or 3-fold increase in the concentration of the at least one nitrated alpha-synuclein protein in the CSF relative to the reference level.

In some embodiments of any of the foregoing aspects, the expression level of the nitrated alpha-synuclein protein is calculated as the concentration of the nitrated alpha-synuclein protein in the CSF sample divided by the concentration of total alpha-synuclein in the CSF sample. In some embodiments of any of the foregoing aspects, the reference expression level is calculated as the concentration of the nitrated alpha-synuclein protein in a reference sample divided by the concentration of total alpha-synuclein in the reference sample.

In some embodiments of any of the foregoing aspects, the reference sample is a CSF sample from a normal subject that does not have a synucleinopathy.

In some embodiments of any of the foregoing aspects, after administering, the method further includes determining the protein expression level of a nitrated alpha-synuclein protein including a nitration of Y39, Y125, Y133, and/or Y136 in an alpha-synuclein protein in a CSF sample obtained from the subject, wherein a decrease (e.g., a statistically significant decrease, e.g., a statistically significant decrease of at least a 10%, 25%, 50%, 100%, 2-fold, or 3-fold increase) in the expression level of the nitrated alpha-synuclein protein relative to the expression level before administration identifies the subject as having responded to treatment.

In some embodiments of any of the foregoing aspects, after administering, the method further includes determining the protein expression level of a nitrated alpha-synuclein protein including a nitration of Y39, Y125, Y133, and/or Y136 in an alpha-synuclein protein in a CSF sample obtained from the subject, wherein an increase (e.g., a statistically significant increase, e.g., a statistically significant increase of at least a 10%, 25%, 50%, 100%, 2-fold, or 3-fold increase) in the expression level of the nitrated alpha-synuclein protein relative to the expression level before administration identifies the subject as in need of further treatment. In some embodiments, the method further includes administering to the subject a therapeutically effect amount of a cognition-enhancing agent (e.g., donepezil, rivastigmine tartrate, galantamine HBr, memantine, and modafinil), an antidepressant agent (e.g., sertraline, fluoxetine, citalopram, escitalopram, paroxetine, and fluvoxamine), a dopamine promoter (e.g., agonist e.g., selegiline, pramipexole and levodopa (L-DOPA)), an anti-tremor agent (e.g., propranolol, primidone, gabapentin, and topiramate), and/or a neuroprotective agent (e.g., a gene encoding all or a portion of Kifap3, Bcl-xl, Crmp1, Chk.beta., CALM2, Caly, NPG11, NPT1, Eef1a1, Dhps, Cd151, Morf412, CTGF, LDH-A, Atl1, NPT2, Ehd3, Cox5b, Tuba1a, gamma-actin, Rpsa, NPG3, NPG4, NPG5, NPG6, NPG7, NPG8, NPG9, or NPG1; gangliosides, topiramate, riluzole, methylprednisolone, rivstigmine, selegiline, cilostazol, rasagiline, tenocyclidine, 7-nitroindazole, N-(3-propylcarbamoyloxirane-2-carbonyl)-isoleucyl-proline, huperzine A, SGS-742, D-JNKI-1, nalmefene, ziconotide, dexanabinol, remacemide, clomethiazole, propentofylline, Z-Val-Ala-Asp fluoromethyl ketone, piracetam, epigallocatechin gallate, vinpocetine, tempol, butylphthalide, eliprodil, tirilazad, nefiracetam, gacyclidine, nizofenone, meclofenoxate, linopiridine, fosfructose, methylprednisolone hemisuccinate, dextrorphan, ebselen, almitrine, brimapitide, edaravone, edaravone, minocycline, epoetin-β, trafermin, filgrastim, eicosapentaenoic acid, and/or pioglitazone).

In another aspect, the invention features a kit for diagnosing a synucleinopathy in a subject, the kit including: (a) probes or antibodies capable of determining the level of one or more of a nitrated alpha-synuclein protein (e.g., 1, 2, 3, or 4 nitrations per alpha-synuclein protein (e.g., nY39/nY136) and/or 1, 2, 3, or 4 nitrated alpha-synuclein proteins (e.g., alpha-synuclein nY36 and alpha-synuclein nY136)) in a CSF sample from the subject; and optionally (b) instructions for use of the probes or antibodies to determine the level of one or more nitrated alpha-synuclein protein in the CSF sample from the subject, wherein an increase (e.g., a statistically significant increase, e.g., a statistically significant increase of at least a 10%, 25%, 50%, 100%, 2-fold, or 3-fold increase) in the level of one or more of the nitrated alpha-synuclein protein relative to a respective reference level indicates that the subject is likely to have a synucleinopathy.

In some embodiments, the one or more nitrated alpha-synuclein protein is nY39 and nY136. For example, in some embodiments, the one or more nitrated alpha-synuclein protein is nY39. In some embodiments, the one or more nitrated alpha-synuclein protein is nY136.

In some embodiments of any of the foregoing aspects, the synucleinopathy is PD or a subtype of the disease thereof, DLB, or MSA. In some embodiments, the subtype of the disease thereof is a prodromal stage of PD. In some embodiments, the synucleinopathy is not Alzheimer's disease.

### Definitions

In this application, unless otherwise clear from context, (i) the term “a” may be understood to mean “at least one”; (ii) the term “or” may be understood to mean “and/or”; and (iii) the terms “including” and “including” may be understood to encompass itemized components or steps whether presented by themselves or together with one or more additional components or steps.

As used herein, the terms “about” and “approximately” refer to a value that is within 10% above or below the value being described. For example, the term “about 5%” indicates a range of from 4.5% to 5.5%.

As used herein, the terms “administration” or “administering” refer to the administration of a composition (e.g., a compound as described herein e.g., a cognition-enhancing agent, an antidepressant agent, a dopamine promoter (e.g., agonist), an anti-tremor agent, and/or a neuroprotective agent) to a subject or system. Administration to an animal subject (e.g., to a human) may be by any appropriate route. Exemplary routes of administration are described herein and below (e.g., intracerebroventricular (ICV) injection, intrathecal (IT) injection, intraparenchymal (IP) injection, intravenous (IV) injection, and stereotactic injection). Administration may be systemic or local.

The term “alpha-synuclein” refers to proteins whose amino acid sequence includes or consists of an amino acid sequence of a naturally occurring wild-type alpha-synuclein protein as well as proteins whose amino acid sequence includes or consists of an amino acid sequence of a naturally occurring mutant alpha-synuclein protein. For example, as used herein, the term alpha-synuclein encompasses full-length, unprocessed alpha-synuclein, as well as any form of alpha-synuclein resulting from processing in the cell (e.g., truncations e.g., C-terminal truncated forms of alpha-synculein and N-terminal truncated forms of alpha-synculein), as well as any naturally occurring variants of alpha-synuclein (e.g., truncations, splice variants, and allelic variants). Alpha-synuclein is also referred to as synuclein alpha (SNCA). Human alpha-synuclein has NCBI Gene ID NO 6622. Alpha-synuclein is considered an intrinsically disordered protein. Exemplary alpha-synuclein amino acid sequences are provided in SEQ ID NOs: 1-8. As used herein, alpha-synuclein may refer to the amino acid of SEQ ID NOs: 1-8, any truncated form thereof, or a truncated form of alpha-synuclein including, but not limited to those described in U.S. Pat. No. 7,306,945, incorporated herein by reference in its entirety.

The nomenclature for describing nitration uses the format “nXB,” where “n” designates the presence of nitration, “X” designates the amino acid found in the wild type variant of the alpha-synuclein protein, and “B” designates the number of the amino acid within the alpha-synuclein peptide chain. For example, a nitrated variant described herein contains a nitration described as nY39, which corresponds to a post-translational nitration at the alpha-synuclein protein at amino acid residue #39, in which a tyrosine is nitrated. The nitrations (e.g., 1, 2, 3, or 4 nitrations per alpha-synuclein protein (e.g., nY39/nY136) and/or 1, 2, 3, or 4 nitrated alpha-synuclein proteins (e.g., alpha-synuclein nY36 and alpha-synuclein nY136)) described herein appear in the context of a wild-type alpha-synuclein protein, for example corresponding to the amino acid sequence of SEQ ID NOs: 1-8, below, or a truncation thereof:

(SEQ ID NO: 1)
MDVFMKGLSKAKEGVVAAAEKTKQGVAEAAGKTKEGVLYVGSKTK
EGVVHGVATVAEKTKEQVTNVGGAVVTGVTAVAQKTVEGAGSIAA
ATGFVKKDQLGKNEEGAPQEGILEDMPVDPDNEAYEMPSEEGYQD
YEPEA;
(SEQ ID NO: 2)
MDVFMKGLSKAKEGVVAAAEKTKQGVAEAAGKTKEGVLYVGSKTK
EGVVHGVATVAEKTKEQVTNVGGAVVTGVTAVAQKTVEGAGSIAA
ATGFVKKDQLGKEGYQDYEPEA;
(SEQ ID NO: 3)
MDVFMKGLSKAKEGVVAAAEKTKQGVAEAAGKTKEGVLYVVAEKT
KEQVTNVGGAVVTGVTAVAQKTVEGAGSIAAATGFVKKDQLGKNE
EGAPQEGILEDMPVDPDNEAYEMPSEEGYQDYEPEA;
(SEQ ID NO: 4)
MDVFMKGLSKAKEGVVAAAEKTKQGVAEAAGKTKEGVLYVGSKTK
EGVVHGVATVAEKTKEQVTNVGGAVVTGVTAVAQKTVEGAGSIAA
ATGFVKKDQLGKSSLRNECSPEPAASTDQSQNEEGAPQEGILEDM
PVDPDNEAYEMPSEEGYQDYEPEA;
(SEQ ID NO: 5)
MDVFMKGLSKAKEGVVAAAEKTKQGVAEAAGKTKEGVLYVGSKTK
EGVVHGVATVAEKTKEQVTNVGGAVVTGVTAVAQKTVEGAGSIAA
ATGFVKKDQLGKNEEGAPQEGILEDMPVDPDNEAYEMPSEPPCCE
EAQATWRGHAMCTCSS;
(SEQ ID NO: 6)
MDVFMKGLSKAKEGVVAAAEKTKQGVAEAAGKTKEGVLYVGSKTK
EGVVHGVATVAEKTKEQVTNVGGAVVTGVTAVAQKTVEGAGSIAA
ATGFVKKDQLGKVWLCTFCVTFISW;
(SEQ ID NO: 7)
MDVFMKGLSKAKEGVVAAAEKTKQGVAEAAGKTKEGVLYVGSKTK
EGVVHGVATVAEKTKEQVTNV;
and
(SEQ ID NO: 8)
MDVFMKGLSKAKEGVVAAAEKTKQGVAEAAGKTKEGVLYVVAEKT
KEQVTNVGGAVVTGVTAVAQKTVEGAGSIAAATGFVKKDQLGKEG
YQDYEPEA;

Thus, references herein to nitrations made “in an alpha-synuclein protein” indicate that the alpha-synuclein protein containing one or more nitrations is in the context of an alpha-synuclein protein or a variant (e.g. truncation) thereof with the above-referenced sequence (e.g., the reference sequence serves as the backbone for the alpha-synuclein protein with the indicated nitration(s)).

The term “changed,” as used herein, refers to an observable difference in the level of a marker in a subject (e.g., in a sample from the subject), as determined using techniques and methods known in the art for the measurement of the marker. A marker level that is changed in a subject may result in a difference of at least 1% (e.g., at least 5%, 10%, 15%, 20%, 25%, 30%, 35%, 40%, 45%, 50%, 55%, 60%, 65%, 70%, 75%, 80%, 85%, 90%, 95%, or 100%, or at least 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold) more than a reference level (e.g., a level from a healthy subject) (e.g., up to 100% or up to 100-fold relative to the reference level). In some embodiments, the change is an increase in the level of a marker (e.g., nitrated alpha-synuclein) in a subject. Increasing the marker level in a subject may result in an increase of at least about 1% (e.g., at least 5%, 10%, 15%, 20%, 25%, 30%, 35%, 40%, 45%, 50%, 55%, 60%, 65%, 70%, 75%, 80%, 85%, 90%, 95%, 98%, 99%, or 100%, or at least 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold) relative to the reference level (e.g., up to 100% or up to 100-fold relative to the reference level).

By “determining the level of a protein” is meant the detection of a protein, by methods known in the art either directly or indirectly. As used herein, determining entails “directly determining” and “indirectly determining.” “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, single molecule detection (e.g., the SIMOA® assay), western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners.

The terms “level of expression” or “expression level” in general are used interchangeably and generally refer to the amount of a biomarker (e.g., nitrated alpha-synuclein) in a biological sample (e.g., a CSF sample). “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., post-translational modification of a polypeptide) shall also be regarded as expressed whether they originate from a transcript generated by alternative splicing or a degraded transcript, or from a post-translational processing of the polypeptide, e.g., by proteolysis.

The terms “diagnose,” “diagnosing,” or “diagnosis” are used herein to refer to the identification or classification of a molecular or pathological state, disease, or condition (e.g., PD, multiple system atrophy (MSA), and dementia with Lewy Bodies (DLB)). For example, “diagnosis” may refer to identification of a particular subtype of PD (e.g., prodromal PD).

The phrase “identifying a patient” or “identifies a patient” as used herein, refers to using the information or data generated relating to the level of at least one nitrated alpha-synuclein protein selected from the group consisting of an alpha-synuclein protein nitrated at tyrosine residue 39 (nY39), nY125, nY133, and nY136 in a CSF sample of a patient to identify or select the patient as more likely to benefit or less likely to benefit from a therapy including a cognition-enhancing agent, an antidepressant agent, a dopamine promoter (e.g., agonist), an anti-tremor agent, and/or a neuroprotective agent. The information or data used or generated may be in any form, written, oral or electronic. In some embodiments, using the information or data generated includes communicating, presenting, reporting, storing, sending, transferring, supplying, transmitting, dispensing, or combinations thereof. In some embodiments, communicating, presenting, reporting, storing, sending, transferring, supplying, transmitting, dispensing, or combinations thereof are performed by a computing device, analyzer unit, or combination thereof. In some further embodiments, communicating, presenting, reporting, storing, sending, transferring, supplying, transmitting, dispensing, or combinations thereof are performed by a laboratory or medical professional. In some embodiments, the information or data includes a comparison of the level of at least one of nY39, nY125, nY133, and nY136 to a reference level. In some embodiments, the information or data includes an indication that at least one of nY39, nY125, nY133, and nY136 is present or absent in a CSF sample. In some embodiments, the information or data includes an indication that the patient is more likely or less likely to respond to a therapy including a cognition-enhancing agent, an antidepressant agent, a dopamine promoter (e.g., agonist), an anti-tremor agent, and/or a neuroprotective agent.

By “level” is meant a level of a protein, as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, ng/ml) or percentage relative to total protein in a sample.

As used herein, the terms “effective amount,” “therapeutically effective amount,” and “a “sufficient amount” of a cognition-enhancing agent, an antidepressant agent, a dopamine promoter (e.g., agonist), an anti-tremor agent, and/or a neuroprotective agent (e.g., in a subject) described herein refer to a quantity sufficient to, when administered to the subject, including a human, effect beneficial or desired results, including clinical results, and, as such, an “effective amount” or synonym thereto depends on the context in which it is being applied. For example, in the context of treating PD, it is an amount of the agent that reduces the motor and/or cognitive symptoms sufficient to achieve a treatment response as compared to the response obtained without administration of the agent. The amount of a given agent that reduces a symptom of PD, MSA, or DLB will vary depending upon various factors, such as the given agent, the pharmaceutical formulation, the route of administration, the subtype of the pathology (e.g., prodromal PD), the identity of the subject (e.g., age, sex, and/or weight) or host being treated, and the like, but can nevertheless be routinely determined by one of skill in the art. Also, as used herein, a “therapeutically effective amount” of a cognition-enhancing agent, an antidepressant agent, a dopamine promoter (e.g., agonist), an anti-tremor agent, and a neuroprotective agent of the present disclosure is an amount which results in a beneficial or desired result in a subject as compared to a control. As defined herein, a therapeutically effective amount a cognition-enhancing agent, an antidepressant agent, a dopamine promoter (e.g., agonist), an anti-tremor agent, and a neuroprotective agent of the present disclosure may be readily determined by one of ordinary skill by routine methods known in the art. Dosage regimen may be adjusted to provide the optimum therapeutic response.

By a “reference” is meant any useful reference used to compare protein levels. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal CSF sample from a subject not having a synucleinopathy; a sample from a subject that is diagnosed with a synucleinopathy, but not yet treated with a compound described herein; or a sample from a subject that has been treated by a compound described herein. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having PD, MSA, or DLB or a subtype of the disease thereof; a subject that has been treated with a compound described herein. In preferred embodiments, the reference sample, standard, or level is matched to the sample subject sample by at least one of the following criteria: age, weight, sex, disease stage, and overall health. A standard curve of levels of a purified protein, e.g., any described herein, within the normal reference range can also be used as a reference.

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

A “synucleinopathy” is a disorder characterized by misfolding and/or abnormal accumulation of aggregates of alpha-synuclein in the central nervous system (e.g., in neurons or glial cells). Exemplary, non-limiting synucleinopathies include PD, dementia with Lewy bodies (DLB), multiple system atrophy (MSA), pure autonomic failure, incidental Lewy body disease, pantothenate kinase-associated neurodegeneration, Alzheimer's disease, Down's Syndrome, Gaucher disease, or the Parkinsonism-dementia complex of Guam.

As used herein, the terms “treat,” “treated,’ or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

The details of one or more embodiments of the invention are set forth in the description below. Other features, objects, and advantages of the invention will be apparent from the description and from the claims.

### BRIEF DESCRIPTION OF THE DRAWINGS

FIG. 1 is an experimental schematic illustrating a method to determine the presence and/or level of a nitration at a particular amino acid residue of alpha-synuclein (a-syn) based upon the specific binding of a bead (e.g, a magnetic bead) coupled with an antibody, as well as a biotinylation signal (e.g., a biotinylated detection antibody) and marker (e.g., streptavidin-beta-gelactosidase) to a protein biomarker (e.g., nitrated alpha-synuclein e.g., nY39, nY125, nY133, and nY136).

FIGS. 2A-2B are a set of graphs illustrating the comparable levels of total alpha-synuclein in a sample of the cerebrospinal fluid (CSF) of patients with Parkinson's disease (PD), as compared to healthy controls (HC). FIG. 2A is a standardization plot of the concentration of detected total alpha-synuclein in response to the average number of enzymes per bead (AEB) in a single molecule detection array, as described in FIG. 1. FIG. 2B is a graph illustrating the comparable level of total alpha-synuclein in a sample of the CSF in patients with PD, as compared to HC. Abbreviation: LLOQ, lower limit of quantification.

FIGS. 3A-3C are a set of graphs illustrating the elevated level of alpha-synuclein nitration at tyrosine 39 (nY39) in a sample of the CSF of patients with PD, as compared to HC. FIG. 3A is a standardization plot of the concentration of detected nY39 in response to the AEB in a single molecule detection array, as described in FIG. 1. FIG. 3B and FIG. 3C, respectively, are graphs illustrating the elevated total and percent level of nY39 in a sample of the CSF in patients with PD, as compared to HC. Abbreviation: LLOQ, lower limit of quantification.

FIGS. 4A-4C are a set of graphs illustrating the elevated level of alpha-synuclein nitration at tyrosine 136 (nY136) in a sample of the CSF of patients with PD, as compared to HC. FIG. 3A is a standardization plot of the concentration of detected nY136 in response to the AEB in a single molecule detection array, as described in FIG. 1. FIG. 3B and FIG. 3C, respectively, are graphs illustrating the elevated total or percent level of nY136 in a sample of the CSF in patients with PD, as compared to HC. Abbreviation: LLOQ, lower limit of quantification.

FIG. 5 is a graph illustrating the elevated levels of nY39 and nY136 in CSF samples of exemplary patients with PD (e.g., “PD 990194”), as compared to HC (e.g., “7554”).

FIGS. 6A-6E are a set of graphs comparing healthy controls (HC), Parkinson's disease (PD), and Alzheimer's disease (AD) subjects as described in Example 5. FIG. 6A is a graph showing similar levels of alpha-synuclein in the CSF of (HC), (PD), and (AD) subjects (HC: n=10, avg 1.40 ng/ml; PD: n=10, avg 1.28 ng/ml; AD: n=10, avg 1.78 ng/ml). FIG. 6B and FIG. 6C, respectively, are graphs illustrating the elevated total or percent level of nY39 in a sample of the CSF in patients with PD, as compared to AD and HC (n=10). FIG. 6D and FIG. 6E, respectively, are graphs illustrating the elevated total or percent level of nY136 in a sample of the CSF in patients with PD, as compared to AD and HC (n=10). These data demonstrate that nitrated alpha-synuclein (nY39 and nY136) is not elevated in AD CSF, but is elevated for PD.

### DETAILED DESCRIPTION

We have discovered that a determination of nitrated alpha-synuclein (e.g., nY39, nY125, nY133, and nY136) in a sample taken from the cerebrospinal fluid (CSF) in a subject is an effective measure for the diagnosis (e.g., identification) and/or treatment of a patient having a synucleinopathy.

### Synucleinopathies

Synucleinopathies are characterized by deposition of intracellular protein aggregates that are microscopically visible as Lewy bodies and/or Lewy neurites, where the protein alpha-synuclein is the major component. Synucleinopathies frequently have degeneration of the dopaminergic nigrostriatal system, responsible for the core motor deficits in Parkinsonism (rigidity, bradykinesia, resting tremor). Several non-motor signs and symptoms are thought to precede motor symptoms in Parkinson's disease and other synucleinopathies. Such early signs include, for example, REM sleep behavior disorder and loss of smell and constipation.

Synucleinopathies include Parkinson's disease (PD) (including idiopathic and inherited forms of Parkinson's disease as well as prodromal PD) and Diffuse Lewy Body (DLB) disease (also known as Dementia with Lewy Bodies (DLB), multiple system atrophy (MSA; e.g., Olivopontocerebellar Atrophy, Striatonigral Degeneration, and Shy-Drager Syndrome)), Lewy body variant of Alzheimer's disease, combined Alzheimer's and Parkinson disease, and pure autonomic failure. In some embodiments, the synucleinopathy is not Alzheimer's disease.

In some embodiments, the synucleinopathy is PD. In some embodiments, the PD is a subtype of the disease thereof (e.g., prodromal PD). In some embodiments, the synucleinopathy is DLB. In some embodiments, the synucleinopathy is MSA.

### Methods

### A. Diagnostic Methods

Provided herein are methods for diagnosing a synucleinopathy (e.g., DLB, MSA, PD or a subtype of the disease thereof e.g., prodromal PD) in a subject. Any of the methods may be based on the protein expression level of a nitrated alpha-synuclein biomarker provided herein, for example, an increased expression of one or more of nY39, nY125, nY133, and nY136. Any of the methods may further include administering to the subject a cognition-enhancing agent, an antidepressant agent, a dopamine promoter (e.g., agonist), an anti-tremor agent, and/or a neuroprotective agent.

The invention features a method of diagnosing a synucleinopathy (e.g., DLB, MSA, PD or a subtype of the disease thereof e.g., prodromal PD) in a subject, the method including determining the expression level of at least one nitrated alpha-synuclein protein (e.g., 1, 2, 3, or 4 nitrations per alpha-synuclein protein (e.g., nY39/nY136) and/or 1, 2, 3, or 4 nitrated alpha-synuclein proteins (e.g., alpha-synuclein nY36 and alpha-synuclein nY136)) selected from the group consisting of nY39, nY125, nY133, and nY136 in an alpha-synuclein protein in a CSF sample obtained from the subject; and comparing the expression level of the at least one nitrated alpha-synuclein protein to a reference expression level of the at least one nitrated alpha-synuclein protein, wherein an increase in the expression level of the at least one nitrated alpha-synuclein protein in the sample relative to the reference expression level of the at least one nitrated alpha-synuclein protein identifies a subject having a synucleinopathy (e.g., DLB, MSA, PD or a subtype of the disease thereof e.g., prodromal PD).

The invention further provides a method of identifying a subject having a synucleinopathy (e.g., DLB, MSA, PD or a subtype of the disease thereof e.g., prodromal PD), the method including determining the expression level of at least one nitrated alpha-synuclein protein (e.g., 1, 2, 3, or 4 nitrations per alpha-synuclein protein (e.g., nY39/nY136) and/or 1, 2, 3, or 4 nitrated alpha-synuclein proteins (e.g., alpha-synuclein nY36 and alpha-synuclein nY136)) selected from the group consisting of nY39, nY125, nY133, and nY136 in an alpha-synuclein protein in a sample obtained from the subject; comparing the expression level of the at least one nitrated alpha-synuclein protein to a reference expression level of the at least one nitrated alpha-synuclein protein; wherein an increase in the expression level of the at least one nitrated alpha-synuclein protein in the sample relative to the reference expression level of the at least one nitrated alpha-synuclein protein identifies a subject having a synucleinopathy (e.g., DLB, MSA, PD or a subtype of the disease thereof e.g., prodromal PD). In some instances, the method includes the step of distinguishing which synucleinopathy the subject has (e.g., PD vs AD).

In any of the preceding methods, the expression level of one or more (e.g., 1, 2, 3, or 4) of alpha-synuclein nY39, nY125, nY133, and nY136 in an alpha-synuclein protein can be determined.

In any of the preceding methods, the expression level of one or more (e.g., 1, 2, 3, 4, 5, 6, 7, 8, 9, 10, 11, or 12) newly identified alpha-synuclein nitration sites can be determined.

The presence and/or expression level/amount of various biomarkers described herein (e.g., alpha-synuclein nY39, nY125, nY133, and nY136) in a CSF sample can be analyzed by a number of methodologies, many of which are known in the art and understood by the skilled artisan, including, but not limited to, a single molecule array (Simoa™) assay, single molecule detection, immunohistochemistry (“IHC”), Western blot analysis, immunoprecipitation, molecular binding assays, ELISA, ELIFA, fluorescence activated cell sorting (“FACS”), MassARRAY, proteomics, biochemical enzymatic activity assays, in situ hybridization, fluorescence in situ hybridization (FISH), polymerase chain reaction (PCR) including quantitative real time PCR (qRT-PCR) and other amplification type detection methods, such as, for example, SISBA, TMA and the like, RNA-seq, microarray analysis, gene expression profiling, and/or serial analysis of gene expression (“SAGE”), as well as any one of the wide variety of assays that can be performed by protein and/or fluid array analysis. In some embodiments, the presence and/or expression level/amount of various biomarkers described herein (e.g., alpha-synuclein nY39, nY125, nY133, and nY136) in a CSF sample is analyzed with a Simoa™ assay.

In any of the preceding methods, the presence and/or expression level/amount of a biomarker (e.g., alpha-synuclein nY39, nY125, nY133, and nY136) can be measured by determining protein expression levels of the biomarker. In certain embodiments, the method includes contacting the biological sample (e.g., CSF) with antibodies that specifically bind to a biomarker (e.g., anti-alpha-synuclein antibodies) under conditions permissive for binding of the biomarker, and detecting whether a complex is formed between the antibodies and biomarker. Such method may be an in vitro method. Any method of measuring protein expression levels known in the art or provided herein may be used. For example, in some embodiments, a protein expression level of a biomarker is determined using a method selected from the group consisting of a Simoa™ assay, single molecule detection, low cytometry (e.g., fluorescence-activated cell sorting (FACS™)), Western blot, enzyme-linked immunosorbent assay (ELISA), immunoprecipitation, immunohistochemistry (IHC), immunofluorescence, radioimmunoassay, dot blotting, immunodetection methods, HPLC, surface plasmon resonance, optical spectroscopy, mass spectrometry, and HPLC. In some embodiments, the protein expression level of the biomarker can be determined in a CSF sample.

In some embodiments of any of the methods, the biomarker is nitrated alpha-synuclein (e.g., nY39, nY125, nY133, and nY136). In some embodiments, the biomarker is nY39. In some embodiments, the biomarker is nY125. In some embodiments, the biomarker is nY133. In some embodiments, the biomarker is nY136. In some embodiments, the biomarker is nY39 and nY136. In some embodiments, presence of nY39, nY125, nY133, and/or nY136 is determined relative to a reference. In some embodiments, the reference is a reference value. In some embodiments, the reference is a reference sample (e.g., a control CSF sample or a CSF sample from non-PD patient, a CSF sample from a non-MSA patient, or a CSF sample from a non-DLB patient).

In some instances, the expression level of the protein in the sample is an average (e.g., mean expression or median expression) of the detected nitrated alpha-synuclein variants, the reference expression level of the nitrated alpha-synuclein variants is an average (e.g., mean expression or median expression) of the nitrated alpha-synuclein variants of the reference, and the average of the nitrated alpha-synuclein variants of the sample is compared to the average of the nitrated alpha-synuclein variants of the reference.

In certain embodiments, the presence and/or expression levels/amount of a biomarker in a first sample is increased or elevated as compared to presence/absence and/or expression levels/amount in a second sample. In certain embodiments, the second sample is a reference sample, reference fluid (e.g., CSF), control sample, or control fluid (e.g., CSF). Additional disclosures for determining the presence/absence and/or expression levels/amount of a nitrated alpha-synuclein variant are described herein. For example, in some embodiments, the expression level of the nitrated alpha-synuclein protein is calculated as the concentration of the nitrated alpha-synuclein protein in the CSF sample divided by the concentration of total alpha-synuclein in the CSF sample. In some embodiments, the reference expression level is calculated as the concentration of the nitrated alpha-synuclein protein in a reference sample divided by the concentration of total alpha-synuclein in the reference sample.

In certain embodiments, a reference sample, reference fluid, control sample, or control fluid is a single sample or combined multiple samples from the same subject or individual that are obtained at one or more different time points than when the test sample is obtained. For example, a reference sample, reference fluid, control sample, or control fluid is obtained at an earlier time point from the same subject or individual than when the test sample is obtained. Such reference samples, reference fluids, control samples, or control fluids may be useful if the reference sample is obtained prior to prodromal PD and the test sample is later obtained when the subject has been diagnosed with prodromal PD.

In certain embodiments, a reference sample, reference fluid, control sample, or control fluid is a combined multiple samples from one or more healthy individuals who are not the patient. In certain embodiments, a reference sample, reference fluid, control sample, or control fluid is a combined multiple samples from one or more individuals with a disease (e.g., PD) who are not the subject or individual. In certain embodiments, a reference sample, reference fluid, control sample, or control fluid is pooled protein samples from normal samples from one or more individuals who are not the patient. In certain embodiments, reference samples, reference fluids, control samples, or control fluids are pooled protein samples from CSF samples from one or more individuals with a disease (e.g., PD) who are not the patient.

In some embodiments of any of the methods, elevated or increased expression refers to an overall increase of about any of 1%, 2%, 3%, 4%, 5%, 6%, 7%, 8%, 9%, 10%, 20%, 30%, 40%, 50%, 60%, 70%, 80%, 90%, 95%, 96%, 97%, 98%, 99% or greater, in the level of biomarker (e.g., nitrated alpha-synuclein protein), detected by standard art-known methods such as those described herein, as compared to reference sample, reference fluid, control sample, or control fluid. In certain embodiments, the elevated expression refers to the increase in expression level/amount of a biomarker in the sample wherein the increase is at least about any of 1.5×, 1.75×, 2×, 3×, 4×, 5×, 6×, 7×, 8×, 9×, 10×, 25×, 50×, 75×, or 100× the expression level/amount of the respective biomarker in reference sample, reference fluid, control sample, or control fluid. In some embodiments, elevated expression refers to an overall increase of greater than about 1.5 fold, about 1.75 fold, about 2 fold, about 2.25 fold, about 2.5 fold, about 2.75 fold, about 3.0 fold, or about 3.25 fold as compared to reference sample, reference fluid, control sample, or control fluid. In some embodiments, the increase is a statistically significant increase (e.g., an increase with a p value of less than or equal to 0.05 (e.g., less than or equal to 0.04, 0.03, 0.02, or 0.01) relative to a reference expression level. In some embodiments, the statistically significant increase is an increase is at least a 10%, 25%, 50%, 100%, 2-fold, or 3-fold increase in the concentration of the at least one nitrated alpha-synuclein protein in the CSF relative to the reference level.

### Therapeutic Methods

The present invention further provides methods for treating a patient suffering from a synucleinopathy (e.g., DLB, MSA, PD or a subtype of the disease thereof e.g., prodromal PD). In some instances, the methods of the invention includes administering to the patient an effective amount of a cognition-enhancing agent, an antidepressant agent, a dopamine promoter (e.g., agonist), an anti-tremor agent, and/or a neuroprotective agent. Any of the cognition-enhancing agents, antidepressant agents, dopamine promoters (e.g., agonists), anti-tremor agents, and neuroprotective agents described herein or otherwise known in the art may be used in the methods. In some instances, the methods involve (i) determining the presence and/or expression level of at least one nitrated alpha-synuclein protein (e.g., nY39, nY125, nY133, and nY136) in a CSF sample obtained from a subject and, following step (i), administering a cognition-enhancing agent, an antidepressant agent, a dopamine promoter (e.g., agonist), an anti-tremor agent, and/or a neuroprotective agent to the patient based on the presence and/or expression level of at least one nitrated alpha-synuclein protein (e.g., nY39, nY125, nY133, and nY136), e.g., using any of the methods described herein or known in the art.

The invention features a method of treating a subject suffering from a synucleinopathy (e.g., DLB, MSA, PD or a subtype of the disease thereof e.g., prodromal PD), the method including determining the expression level of at least one nitrated alpha-synuclein protein (e.g., 1, 2, 3, or 4 nitrations per alpha-synuclein protein (e.g., nY39/nY136) and/or 1, 2, 3, or 4 nitrated alpha-synuclein proteins (e.g., alpha-synuclein nY36 and alpha-synuclein nY136)) selected from the group consisting of nY39, nY125, nY133, and nY136 in an alpha-synuclein protein in a CSF sample obtained from the subject; and comparing the expression level of the at least one nitrated alpha-synuclein protein to a reference expression level of the at least one nitrated alpha-synuclein protein, wherein an increase in the expression level of the at least one nitrated alpha-synuclein protein in the sample relative to the reference expression level of the at least one nitrated alpha-synuclein protein identifies a subject having a synucleinopathy (e.g., DLB, MSA, PD or a subtype of the disease thereof e.g., prodromal PD), and administering to the subject a therapeutically effective amount of a cognition-enhancing agent, an antidepressant agent, a dopamine promoter (e.g., agonist), an anti-tremor agent, and/or a neuroprotective agent.

The invention further provides a method of treating a subject suffering from a synucleinopathy (e.g., DLB, MSA, PD or a subtype of the disease thereof e.g., prodromal PD), wherein the expression level of one or more (e.g., 1, 2, 3, 4, 5, 6, 7, 8, 9, 10, 11, or 12) newly identified nitrated alpha-synuclein proteins is determined. Newly identified alpha-synuclein proteins include an alpha-synuclein protein nitrated at an amino acid residue previously unknown to be nitrated.

In any of the preceding methods, the cognition-enhancing agent, antidepressant agent, dopamine promoter (e.g., agonist), anti-tremor agent, and/or neuroprotective agent may be any cognition-enhancing agent, antidepressant agent, dopamine promoter (e.g., agonist), anti-tremor agent, and/or neuroprotective agent known in the art or described herein.

The compositions used in the methods described herein (e.g., a cognition-enhancing agent, an antidepressant agent, a dopamine promoter (e.g., agonist), an anti-tremor agent, and/or a neuroprotective agent) can be administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularly, mucosally, intrapericardially, intraumbilically, intraocularly, intraorbitally, orally, topically, transdermally, intravitreally (e.g., by intravitreal injection), by eye drop, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered and the severity of the condition, disease, or disorder being treated). In some embodiments, the cognition-enhancing agent, antidepressant agent, dopamine promoter (e.g., agonist), anti-tremor agent, and/or neuroprotective agent is administered intravenously, intramuscularly, subcutaneously, topically, orally, transdermally, intraperitoneally, intraorbitally, by implantation, by inhalation, intrathecally, intraventricularly, or intranasally. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Cognition-enhancing agents, antidepressant agents, dopamine promoters (e.g., agonists), anti-tremor agents, and neuroprotective agents described herein may be formulated, dosed, and administered in a fashion consistent with good medical practice. Factors for consideration in this context include the particular disease subtype being treated (e.g., PD and prodromal PD, MSA, and DLB), the particular mammal being treated, the clinical condition of the individual patient, the cause of the disease, the site of delivery of the agent, the method of administration, the scheduling of administration, and other factors known to medical practitioners.

Exemplary cognition-enhancing agents useful in the methods of the invention include, but are not limited to, donepezil, rivastigmine tartrate, galantamine HBr, memantine, and modafinil.

Exemplary antidepressant agents useful in the methods of the invention include sertraline, fluoxetine, citalopram, escitalopram, paroxetine, and fluvoxamine.

Exemplary anxiolytic agents useful in the methods of the invention include, but are not limited to, alprazolam, chlordiazepoxide, clobazepam, clonazepam, clorazepate, diazepam, estazolam, and flurazepam.

Exemplary antipsychotic agents useful in the methods of the invention include, but are not limited to, aripiprazole, asenapine, cariprazine, clozapine, lurasidone, olanzapine, quetiapine, and risperidone.

Exemplary sedatives useful in the methods of the invention include, but are not limited to, alprazolam, chloral hydrate, chlordiazepoxide, clorazepate, clonazepam, diazepam, and estazolam.

Exemplary dopamine promoters (agonists) useful in the methods of the invention include, but are not limited to, selegiline, pramipexole and levodopa (L-DOPA).

Exemplary anti-tremor agents useful in the methods of the invention include, but are not limited to, propranolol, primidone, gabapentin, and topiramate.

Exemplary neuroprotective agents useful in the methods of the invention include, but are not limited to, a gene encoding all or a portion of Kifap3, Bcl-xl, Crmp1, Chk.beta., CALM2, Caly, NPG11,NPT1, Eef1a1, Dhps, Cd151, Morf412, CTGF, LDH-A, Atl1, NPT2, Ehd3, Cox5b, Tuba1a, gamma-actin, Rpsa, NPG3, NPG4, NPG5, NPG6, NPG7, NPG8, NPG9, or NPG1. Furthermore, other exemplary neuroprotective agents include, but are not limited to, gangliosides, topiramate, riluzole, methylprednisolone, rivstigmine, selegiline, cilostazol, rasagiline, tenocyclidine, 7-nitroindazole, N-(3-propylcarbamoyloxirane-2-carbonyl)-isoleucyl-proline, huperzine A, SGS-742, D-JNKI-1, nalmefene, ziconotide, dexanabinol, remacemide, clomethiazole, propentofylline, Z-Val-Ala-Asp fluoromethyl ketone, piracetam, epigallocatechin gallate, vinpocetine, tempol, butylphthalide, eliprodil, tirilazad, nefiracetam, gacyclidine, nizofenone, meclofenoxate, linopiridine, fosfructose, methylprednisolone hemisuccinate, dextrorphan, ebselen, almitrine, brimapitide, edaravone, edaravone, minocycline, epoetin-β, trafermin, filgrastim, eicosapentaenoic acid, and pioglitazone.

In some embodiments, after administration of a cognition-enhancing agent, antidepressant agent, dopamine promoter (e.g., agonist), anti-tremor agent, and/or neuroprotective agent described herein, the method further includes determining the protein expression level of a nitrated alpha-synuclein protein including a nitration of Y39, Y125, Y133, and/or Y136 in an alpha-synuclein protein in a CSF sample obtained from the subject, wherein an increase in the expression level of the nitrated alpha-synuclein protein relative to the expression level before administration identifies the subject as in need of further treatment. In some embodiments, said subject is further administered a second dose of a cognition-enhancing agent, antidepressant agent, dopamine promoter (e.g., agonist), anti-tremor agent, and/or neuroprotective agent.

### Kits

The invention also features kits including (a) probes or antibodies capable of determining the level of one or more nitrated alpha-synuclein protein in a CSF sample from the subject described herein, and (b) a package insert with instructions to perform any of the methods described herein.

### EXAMPLES

Example 1—Nitrated Alpha-Synuclein is Increased in the Cerebrospinal Fluid of Subjects with Parkinson'S Disease

### SIMOA® Assay Procedure

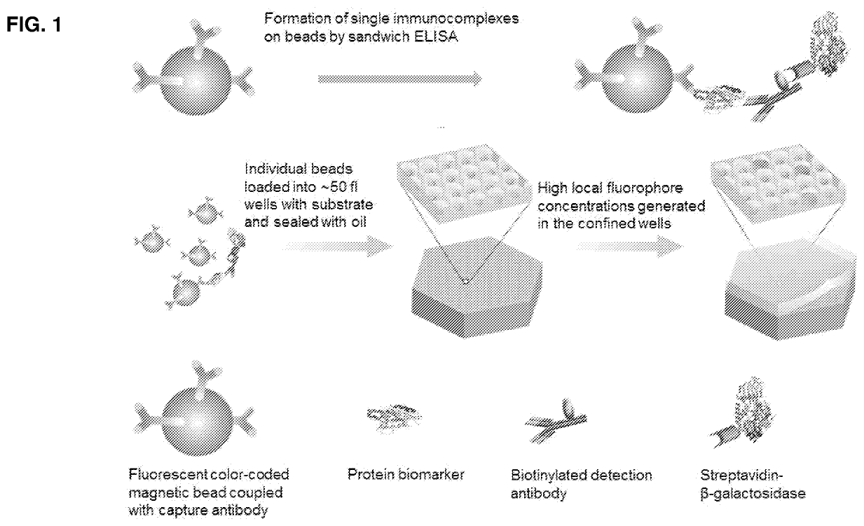

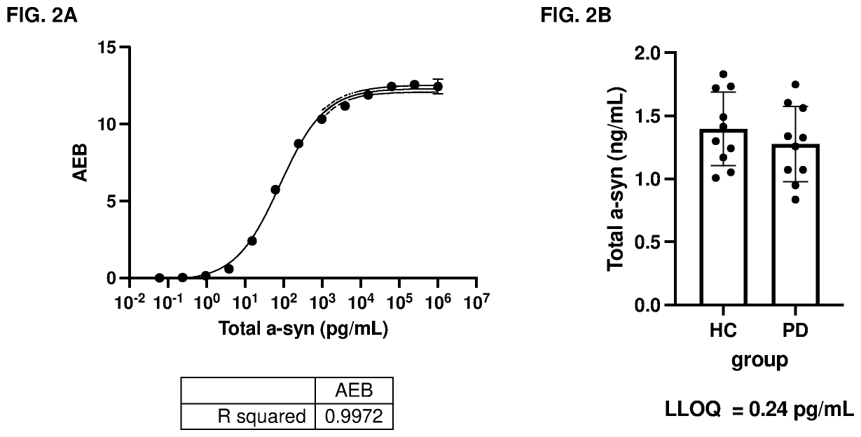

The SIMOA® assay was performed using a monoclonal antibody nSyn12 (Millipore Sigma 36-011) to capture labelled and dissociated nitrated alpha-synuclein onto beads. Briefly, capture beads and antibody were prepared by buffer exchange into the QUANTERIX®-recommended Bead Conjugation Buffer using AMICON® Ultra-0.5 centrifugal filters. Conjugation of the capture antibody to beads was based on 1-ethyl-3-(3-dimethylaminopropyl) carbodiimide (EDAC) chemistry and performed according to QUANTERIX® manual protocol. Beads were further characterized through the bead aggregation protocol on the SIMOA® HD-1 ANALYZER™ (QUANTERIX®) for bead number and aggregation level, as recommended (FIGS. 2A, 3A, and 4A). The detection reagent was the biotinylated-mouse monoclonal anti-alpha-synuclein antibody (BD Biosciences; catalog number 610787) which was used to bind the active site of the enzyme during sample preparation. A two-step protocol, where a sample is co-incubated with capture beads and detection reagent (for 30 min) in a single step, followed by the sequential addition and washing of streptavidin-beta galactosidase (SBG 100 pM) and the fluorescent substrate e.g., RGP (resorufin-β-D-galactopyranoside) for signal generation was utilized. SIMOA® analysis software was used to quantify all samples, using a 4-PL fit of standards with a 1/Y² weighting (FIG. 1).

### Results

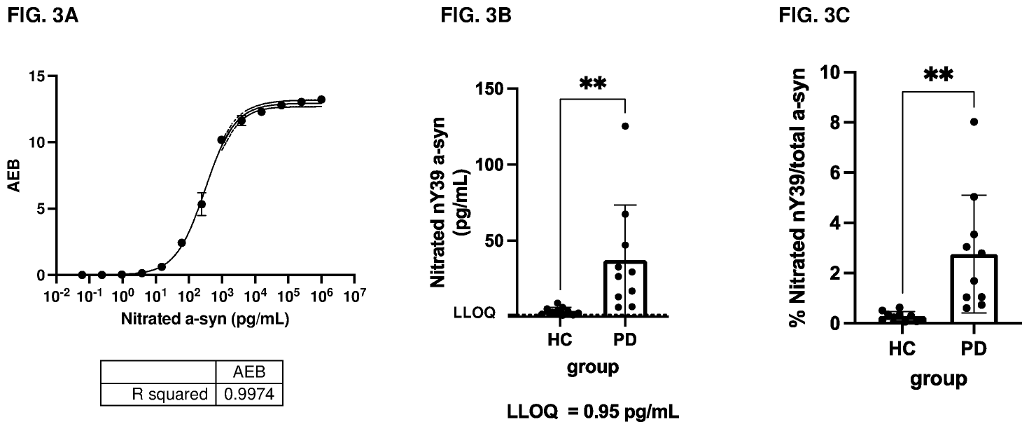

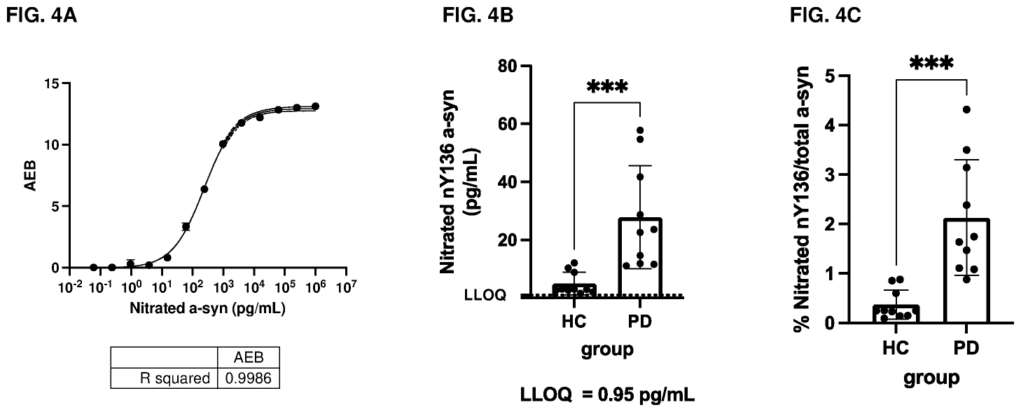

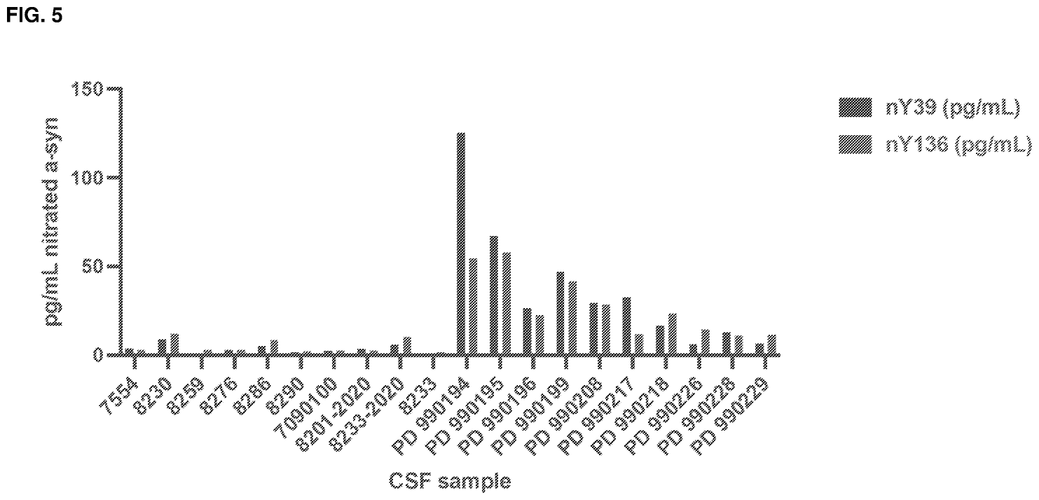

As shown in FIGS. 3B-3C, 4B-4C, and 5, nitrated alpha-synuclein (e.g., nitrated tyrosine 39 (nY39) in FIGS. 3B-3C and nY136 in FIGS. 4B-4C) is significantly elevated in the cerebrospinal fluid (CSF) of patients having Parkinson's disease (PD), as compared to healthy controls (HC). The same trend was observed when individual patients were separated (FIG. 5 and Table 1, below). In contrast, the level of total alpha-synuclein in the CSF was comparable across PD and HC groups (FIG. 2B).

These results demonstrate that nitrated alpha-synuclein protein in the CSF can serve as a diagnostic biomarker for PD or a subtype of the disease thereof (e.g., prodromal PD).

TABLE 1
nY39 alpha-synuclein is increased in the CSF of subjects with Parkinson's disease

| | | | | | MSD | | | SIMOA | | |
|---|---|---|---|---|---|---|---|---|---|---|
| | | | | | Total | nY39 | | Total | nY39 | |
| | | | | | a-syn | a-syn | | a-syn | a-syn | |
| Donor | | Consent | | | (ng/ | (pg/ | % NT/ | (ng/ | (pg/ | % NT/ |
| ID # | DOB | Date | Sex | Diagnosis | mL) | mL) | Total | mL) | mL | Total |
| 7554 | Jan. 17, | Feb. 22, | F | HC | 1.23133015 | | | 1.29890502 | 3.92704658 | 0.3 |
| | 1961 | 2012 | | | | | | | | |
| 8230 | Dec. 9, | Mar. 18, | M | HC | 1.77789288 | | | 1.41369704 | 8.90259443 | 0.6 |
| | 1959 | 2019 | | | | | | | | |
| 8259 | Jun. 15, | Aug. 24, | M | HC | 1.10066083 | | | 1.1704054 | 0.97465142 | 0.1 |
| | 1965 | 2020 | | | | | | | | |
| 8276 | Jul. 26, | Sep. 16, | F | HC | 1.24988374 | | | 1.24301271 | 3.10660321 | 0.2 |
| | 1957 | 2020 | | | | | | | | |
| 8286 | Oct. 29, | Dec. 16, | M | HC | 0.8729307 | | | 1.00771744 | 5.18808603 | 0.5 |
| | 1958 | 2020 | | | | | | | | |
| 8290 | Feb. 17, | Dec. 9, | M | HC | 1.43402101 | | | 1.49028661 | 1.71574838 | 0.1 |
| | 1962 | 2020 | | | | | | | | |
| 7090100 | Dec. 13, | Nov. 11, | M | HC | 7.35190652 | | | 1.83144724 | 2.49327921 | 0.1 |
| | 1949 | 2016 | | | | | | | | |
| 8201- | May 10, | Jul. 10, | F | HC | 1.10066083 | | | 1.05317143 | 3.50439032 | 0.3 |
| 2020 | 1944 | 2020 | | | | | | | | |
| 8233- | Mar. 19, | Jul. 15, | M | HC | 2.44798524 | | | 1.72076002 | 6.03295452 | 0.4 |
| 2020* | 1962 | 2020 | | | | | | | | |
| 8233* | Mar. 19, | Mar. 25, | M | HC | 2.930561 | | | 1.73352274 | 1.03746647 | 0.1 |
| | 1962 | 2019 | | | | | | | | |
| 990194 | Sep. 4, | May 6, | F | PD | 1.9384924 | 318.239912 | 16.4 | 1.56183492 | 125.256963 | 8.0 |
| | 1969 | 2020 | | | | | | | | |
| 990195 | Dec. 11, | May 11, | F | PD | 1.26841054 | 215.39691 | 17.0 | 1.33891925 | 67.4432221 | 5.0 |
| | 1960 | 2020 | | | | | | | | |
| 990196 | Jun. 3, | Jun. 10, | M | PD | 0.73741403 | 114.371079 | 15.5 | 0.94931911 | 26.4469687 | 2.8 |
| | 1953 | 2020 | | | | | | | | |
| 990199 | May 6, | Aug. 25, | M | PD | 1.06305399 | 150.093804 | 14.1 | 1.32808884 | 47.0001872 | 3.5 |
| | 1967 | 2020 | | | | | | | | |
| 990208 | Jan. 31, | Jun. 16, | M | PD | 2.43059501 | 95.3196319 | 3.9 | 1.74867142 | 29.442811 | 1.7 |
| | 1957 | 2020 | | | | | | | | |
| 990217 | Aug. 24, | Sep. 29, | M | PD | 0.69825689 | 158.641558 | 22.7 | 1.07180679 | 32.6761664 | 3.0 |
| | 1948 | 2020 | | | | | | | | |
| 990218 | Jun. 5, | Nov. 4, | M | PD | 2.25603276 | | | 1.60485901 | 16.7947314 | 1.0 |
| | 1950 | 2020 | | | | | | | | |
| 990226 | Sep. 26, | Nov. 17, | F | PD | | | | 0.83562212 | 6.14780407 | 0.7 |
| | 1940 | 2020 | | | | | | | | |
| 990228 | Aug. 6, | Jan. 5, | F | PD | 1.13814235 | | | 1.2574239 | 13.0366126 | 1.0 |
| | 1959 | 2021 | | | | | | | | |
| 990229 | Oct. 6, | Jan. 18, | F | PD | 0.77636649 | | | 1.07226526 | 6.51638216 | 0.6 |
| | 1948 | 2021 | | | | | | | | |

### Example 2—Diagnosis of a Synucleinopathy in Human Subjects by Determination of at Least One Nitrated Alpha-Synuclein Protein in a Cerebrospinal Fluid Sample Obtained from the Subject

Using the methods of the disclosure, a subject (e.g., a human subject), may be diagnosed (e.g., identified) as having a synucleinopathy (e.g., DLB, MSA, PD or a subtype of the disease thereof e.g., prodromal PD). For example, after determining (e.g., with a SIMOA® assay) the expression level of at least one (e.g., two, three, or four) nitrated alpha-synuclein protein selected from the group consisting of nY39, nY125, nY133, and nY136 in an alpha-synuclein protein in a CSF sample obtained from the subject; and comparing the expression level of the at least one nitrated alpha-synuclein protein to a reference level of the at least one nitrated alpha-synuclein protein, wherein an increase in the expression level of the at least one nitrated alpha-synuclein protein in the CSF sample relative to the reference expression level of the at least one nitrated alpha-synuclein protein, a subject may be identified as having a synucleinopathy.

Upon diagnosis, the subject may be administered a therapeutically effect amount of a cognition-enhancing agent (e.g., donepezil, rivastigmine tartrate, galantamine HBr, memantine, and modafinil), an antidepressant agent (e.g., sertraline, fluoxetine, citalopram, escitalopram, paroxetine, and fluvoxamine), a dopamine promoter (e.g., agonist e.g., selegiline, pramipexole and levodopa (L-DOPA)), an anti-tremor agent (e.g., propranolol, primidone, gabapentin, and topiramate), and/or a neuroprotective agent (e.g., a gene encoding all or a portion of Kifap3, Bcl-xl, Crmp1, Chk.beta., CALM2, Caly, NPG11, NPT1, Eef1a1, Dhps, Cd151, Morf412, CTGF, LDH-A, Atl1, NPT2, Ehd3, Cox5b, Tuba1a, gamma-actin, Rpsa, NPG3, NPG4, NPG5, NPG6, NPG7, NPG8, NPG9, or NPG1; gangliosides, topiramate, riluzole, methylprednisolone, rivstigmine, selegiline, cilostazol, rasagiline, tenocyclidine, 7-nitroindazole, N-(3-propylcarbamoyloxirane-2-carbonyl)-isoleucyl-proline, huperzine A, SGS-742, D-JNKI-1, nalmefene, ziconotide, dexanabinol, remacemide, clomethiazole, propentofylline, Z-Val-Ala-Asp fluoromethyl ketone, piracetam, epigallocatechin gallate, vinpocetine, tempol, butylphthalide, eliprodil, tirilazad, nefiracetam, gacyclidine, nizofenone, meclofenoxate, linopiridine, fosfructose, methylprednisolone hemisuccinate, dextrorphan, ebselen, almitrine, brimapitide, edaravone, edaravone, minocycline, epoetin-β, trafermin, filgrastim, eicosapentaenoic acid, and/or pioglitazone).

### Example 3—Treatment of a Synucleinopathy in Human Subjects

Using the compositions and methods of the disclosure, a subject (e.g., a human subject), may be treated for a synucleinopathy (e.g., DLB, MSA, PD or a subtype of the disease thereof e.g., prodromal PD). For example, after determining (e.g., with a SIMOA@ assay) the expression level of at least one (e.g., two, three, or four) nitrated alpha-synuclein protein selected from the group consisting of nY39,nY125, nY133, and nY136 in an alpha-synuclein protein in a CSF sample obtained from the subject; and comparing the expression level of the at least one nitrated alpha-synuclein protein to a reference level of the at least one nitrated alpha-synuclein protein, wherein an increase in the expression level of the at least one nitrated alpha-synuclein protein in the CSF sample relative to the reference expression level of the at least one nitrated alpha-synuclein protein, a subject may be identified as having a synucleinopathy and may be administered a therapeutically effect amount of a cognition-enhancing agent (e.g., donepezil, rivastigmine tartrate, galantamine HBr, memantine, and modafinil), an antidepressant agent (e.g., sertraline, fluoxetine, citalopram, escitalopram, paroxetine, and fluvoxamine), a dopamine promoter (e.g., agonist e.g., selegiline, pramipexole and levodopa (L-DOPA)), an anti-tremor agent (e.g., propranolol, primidone, gabapentin, and topiramate), and/or a neuroprotective agent (e.g., a gene encoding all or a portion of Kifap3, Bcl-xl, Crmp1, Chk.beta., CALM2, Caly, NPG11, NPT1, Eef1a1, Dhps, Cd151, Morf412,CTGF, LDH-A, Atl1, NPT2, Ehd3, Cox5b, Tuba1a, gamma-actin, Rpsa, NPG3, NPG4, NPG5, NPG6,NPG7, NPG8, NPG9, or NPG1; gangliosides, topiramate, riluzole, methylprednisolone, rivstigmine, selegiline, cilostazol, rasagiline, tenocyclidine, 7-nitroindazole, N-(3-propylcarbamoyloxirane-2-carbonyl)-isoleucyl-proline, huperzine A, SGS-742, D-JNKI-1, nalmefene, ziconotide, dexanabinol, remacemide, clomethiazole, propentofylline, Z-Val-Ala-Asp fluoromethyl ketone, piracetam, epigallocatechin gallate, vinpocetine, tempol, butylphthalide, eliprodil, tirilazad, nefiracetam, gacyclidine, nizofenone, meclofenoxate, linopiridine, fosfructose, methylprednisolone hemisuccinate, dextrorphan, ebselen, almitrine, brimapitide, edaravone, edaravone, minocycline, epoetin-β, trafermin, filgrastim, eicosapentaenoic acid, and/or pioglitazone).

### Example 4—Treatment of a Synucleinopathy in Human Subjects

Using the compositions and methods of the disclosure, a subject (e.g., a human subject), may be treated for a synucleinopathy (e.g., DLB, MSA, PD or a subtype of the disease thereof e.g., prodromal PD). For example, after determining (e.g., with a SIMOA® assay) the expression level of at least one (e.g., two, three, or four) nitrated alpha-synuclein protein selected from the group consisting of nY39, nY125, nY133, and nY136 in an alpha-synuclein protein in a CSF sample obtained from the subject; and comparing the expression level of the at least one nitrated alpha-synuclein protein to a reference level of the at least one nitrated alpha-synuclein protein, wherein an increase in the expression level of the at least one nitrated alpha-synuclein protein in the CSF sample relative to the reference expression level of the at least one nitrated alpha-synuclein protein, a subject may be identified as having a synucleinopathy and may be administered a therapeutically effect amount of a cognition-enhancing agent (e.g., donepezil, rivastigmine tartrate, galantamine HBr, memantine, and modafinil), an antidepressant agent (e.g., sertraline, fluoxetine, citalopram, escitalopram, paroxetine, and fluvoxamine), a dopamine promoter (e.g., agonist e.g., selegiline, pramipexole and levodopa (L-DOPA)), an anti-tremor agent (e.g., propranolol, primidone, gabapentin, and topiramate), and/or a neuroprotective agent (e.g., a gene encoding all or a portion of Kifap3, Bcl-xl, Crmp1, Chk.beta., CALM2, Caly, NPG11, NPT1, Eef1a1, Dhps, Cd151, Morf412, CTGF, LDH-A, Atl1, NPT2, Ehd3, Cox5b, Tuba1a, gamma-actin, Rpsa, NPG3, NPG4, NPG5, NPG6, NPG7, NPG8, NPG9, or NPG1; gangliosides, topiramate, riluzole, methylprednisolone, rivstigmine, selegiline, cilostazol, rasagiline, tenocyclidine, 7-nitroindazole, N-(3-propylcarbamoyloxirane-2-carbonyl)-isoleucyl-proline, huperzine A, SGS-742, D-JNKI-1, nalmefene, ziconotide, dexanabinol, remacemide, clomethiazole, propentofylline, Z-Val-Ala-Asp fluoromethyl ketone, piracetam, epigallocatechin gallate, vinpocetine, tempol, butylphthalide, eliprodil, tirilazad, nefiracetam, gacyclidine, nizofenone, meclofenoxate, linopiridine, fosfructose, methylprednisolone hemisuccinate, dextrorphan, ebselen, almitrine, brimapitide, edaravone, edaravone, minocycline, epoetin-β, trafermin, filgrastim, eicosapentaenoic acid, and/or pioglitazone).

After administration of a cognition-enhancing agent, antidepressant agent, dopamine promoter (e.g., agonist), anti-tremor agent, and/or neuroprotective agent described herein, the expression level of at least one (e.g., two, three, or four) nitrated alpha-synuclein protein selected from the group consisting of nY39, nY125, nY133, and nY136 in an alpha-synuclein protein in a CSF sample obtained from the subject is obtained. If an increase in the expression level of the nitrated alpha-synuclein protein relative to the expression level before administration of the treatment is observed, then the subject is identified as a subject in need of further treatment. The subject may be administered a second dose of a cognition-enhancing agent, antidepressant agent, dopamine promoter (e.g., agonist), anti-tremor agent, and/or neuroprotective agent.

### Example 5—Nitrated Alpha-Synuclein is Not Increased in the Cerebrospinal Fluid of Subjects with Alzheimer's Disease

The SIMOA® assay for Y39 synuclein and Y136 synuclein was performed as described in Example 1 on cerebrospinal fluid (CSF) comparing patients having Alzheimer's disease (AD), Parkinson's disease (PD), and healthy controls (HC).

### Results

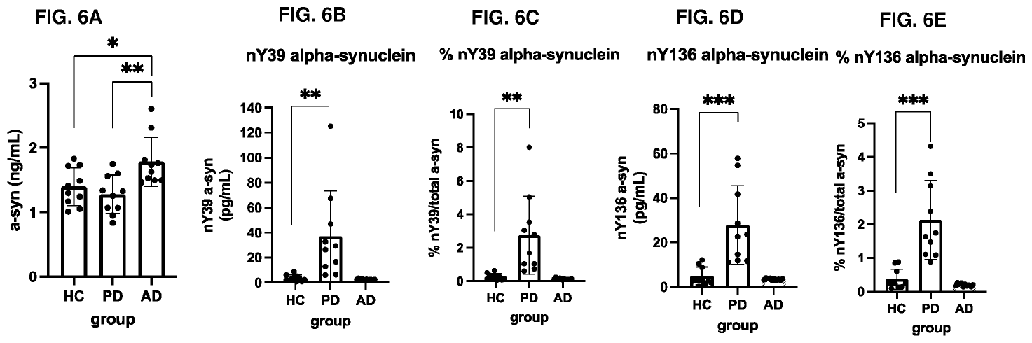

As shown in FIGS. 6A-6E, nitrated alpha-synuclein (nY39 and nY136) is not elevated in AD CSF, but is elevated for PD. FIG. 6A is a graph showing similar levels of alpha-synuclein in the CSF of (HC), (PD), and (AD) subjects (HC: n=10, avg 1.40 ng/ml; PD: n=10, avg 1.28 ng/mL; AD: n=10, avg 1.78 ng/ml). FIG. 6B and FIG. 6C, respectively, are graphs illustrating the elevated total or percent level of nY39 in a sample of the CSF in patients with PD, as compared to AD and HC (n=10). FIG. 6D and FIG. 6E, respectively, are graphs illustrating the elevated total or percent level of nY136 in a sample of the CSF in patients with PD, as compared to AD and HC (n=10).

These results demonstrate that nitrated alpha-synuclein protein in the CSF can serve as a diagnostic biomarker for PD that is specific for PD vs AD, and useful for such a differential diagnosis.

### Other Embodiments

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are in the claims.

What is claimed is:

1. A method of diagnosing a synucleinopathy in a subject, the method comprising:
(a) determining the expression level of at least one nitrated alpha-synuclein protein selected from the group consisting of an alpha-synuclein protein nitrated at tyrosine residue 39 (nY39), nY125, nY133, and nY136 in an alpha-synuclein protein in a cerebrospinal fluid (CSF) sample obtained from the subject; and
(b) comparing the expression level of the at least one nitrated alpha-synuclein protein to a reference level of the at least one nitrated alpha-synuclein protein, wherein an increase in the expression level of the at least one nitrated alpha-synuclein protein in the CSF sample relative to the reference expression level of the at least one nitrated alpha-synuclein protein identifies a subject having a synucleinopathy.

2. The method of claim 1, wherein the expression level of at least two nitrated alpha-synuclein proteins selected from the group consisting of nY39, nY125, nY133, and nY136 in a CSF sample obtained from the subject are determined.

3. The method of claim 1 or 2, wherein a statistically significant increase in the expression level of the at least one nitrated alpha-synuclein protein in the CSF sample relative to the reference expression level of the at least one nitrated alpha-synuclein protein identifies a subject having a synucleinopathy.

4. The method of any one of claims 1-3, wherein the statistically significant increase is an increase is at least a 10%, 25%, 50%, 100%, 2-fold, or 3-fold increase in the concentration of the at least one nitrated alpha-synuclein protein in the CSF relative to the reference level.

5. The method of any one of claims 1-4, wherein the expression level of the nitrated alpha-synuclein protein is calculated as the concentration of the nitrated alpha-synuclein protein in the CSF sample divided by the concentration of total alpha-synuclein in the CSF sample.

6. The method of any one of claims 1-5, wherein the reference expression level is calculated as the concentration of the nitrated alpha-synuclein protein in a reference sample divided by the concentration of total alpha-synuclein in the reference sample.

7. The method of claim 6, wherein the reference sample is a CSF sample from a normal subject that does not have a synucleinopathy.

8. The method of claim 2, wherein:
(a) the expression level of at least two nitrated alpha-synuclein proteins is an average of the at least two nitrated alpha-synuclein protein of the CSF sample;
(b) the reference expression level of the at least two nitrated alpha-synuclein proteins are an average of the at least two nitrated alpha-synuclein proteins of the reference; and
(c) the average of the at least two nitrated alpha-synuclein proteins of the CSF sample are compared to the average of the at least two nitrated alpha-synuclein proteins of the reference.

9. The method of claim 2, wherein the at least two nitrated alpha-synuclein proteins are nY39 and nY136.

10. The method of claim 1, wherein the nitrated alpha-synuclein is nY39.

11. The method of claim 1, wherein the nitrated alpha-synuclein is nY136.

12. The method of claim 1, wherein the one nitrated alpha-synuclein protein is nY39 and nY136.

13. The method of any one of claims 1-12, wherein the synucleinopathy is Parkinson's disease (PD) or a subtype of the disease thereof, dementia with Lewy Bodies (DLB), or Multiple-System Atrophy (MSA)

14. The method of claim 13, wherein the subtype of the disease thereof is a prodromal stage of PD.

15. The method of any one of claims 1-14, wherein the method further comprises administering to the subject an effective amount of a cognition-enhancing agent, an antidepressant agent, a dopamine promoter, an anti-tremor agent, and/or a neuroprotective agent.

16. The method of any one of claims 1-6 and 8-15, wherein the reference expression level is the mean level of expression of the at least one nitrated alpha-synuclein protein in a population of subjects.

17. The method of any one of claims 1-16, wherein the expression level is a protein expression level.

18. The method of claim 17, wherein the protein expression level is determined by single molecule detection, western blot, enzyme-linked immunoassay (ELISA), immunohistochemistry, or mass spectrometry.

19. A method of identifying a subject having a synucleinopathy, the method comprising determining the protein level of alpha-synuclein comprising a nitration of Y39, Y125, Y133, and/or Y136 in an alpha-synuclein protein in a CSF sample obtained from the subject, wherein the presence of nY39, nY125, nY133, and/or nY136 identifies the subject as having a synucleinopathy.

20. The method of claim 19, wherein the protein expression level is determined by single molecule detection, western blot, ELISA, immunohistochemistry, or mass spectrometry.

21. A method of diagnosing a synucleinopathy in a subject, the method comprising determining the protein level of alpha-synuclein comprising a nitration of Y39, Y125, Y133, and/or Y136 in an alpha-synuclein protein in a CSF sample obtained from the subject, wherein the presence of nY39, nY125, nY133, and/or nY136 identifies the subject as having a synucleinopathy.

22. The method of claim 21, wherein the protein expression level is determined by single molecule detection, western blot, ELISA, immunohistochemistry, or mass spectrometry.

23. The method of any one of claims 19-22, wherein the presence of nY39 identifies the subject as having a synucleinopathy.

24. The method of any one of claims 19-23, wherein the presence of nY136 identifies the subject as having a synucleinopathy.

25. The method of any one of claims 19-24, wherein the presence of nY39 and nY136 identifies the subject as having a synucleinopathy.

26. The method of any one of claims 19-25, further comprising administering to the subject a therapeutically effect amount of a cognition-enhancing agent, an antidepressant agent, a dopamine promoter, an anti-tremor agent, and/or a neuroprotective agent.

27. A method of treating a subject having a synucleinopathy, the method comprising administering to the subject a therapeutically effective amount of a cognition-enhancing agent, an antidepressant agent, a dopamine promoter, an anti-tremor agent, and/or a neuroprotective agent, wherein the expression level of a nitrated alpha-synuclein protein having at least one of the following tyrosine residues Y39, Y125, Y133, and Y136 in an alpha-synuclein protein nitrated in a CSF sample obtained from the subject has been determined to be increased relative to a reference expression level of the at least one nitrated alpha-synuclein protein.

28. The method of claim 27, wherein a nitrated alpha-synuclein protein having at least two of the following tyrosine residues Y39, Y125, Y133, and Y136 in an alpha-synuclein protein nitrated in a CSF sample obtained from the subject has been determined.

29. The method of claim 27 or 28, wherein the expression level of a nitrated alpha-synuclein protein having the tyrosine residue Y39 nitrated in a CSF sample obtained from the subject has been determined.

30. The method of any one of claims 27-29, wherein the expression level of a nitrated alpha-synuclein protein having the tyrosine residue Y136 nitrated in a CSF sample obtained from the subject has been determined.

31. The method of any one of claims 28-30, wherein the expression level of a nitrated alpha-synuclein protein having the tyrosine residues Y39 and Y136 nitrated in a CSF sample obtained from the subject have been determined.

32. A method of treating a subject having a synucleinopathy, the method comprising:
(a) determining the protein expression level of a nitrated alpha-synuclein protein comprising a nitration of Y39, Y125, Y133, and/or Y136 in an alpha-synuclein protein in a CSF sample obtained from the subject, wherein an increase in the expression level of the nitrated alpha-synuclein protein relative to a reference expression level identifies the subject as having a synucleinopathy; and
(b) administering to the subject a therapeutically effective amount of a cognition-enhancing agent, an antidepressant agent, a dopamine promoter, an anti-tremor agent, and/or a neuroprotective agent.

33. The method of claim 32, wherein the nitrated alpha-synuclein protein comprises a nitration of Y39.

34. The method of claim 32 or 33, wherein the nitrated alpha-synuclein protein comprises a nitration of Y136.

35. The method of any one of claims 32-34, wherein the nitrated alpha-synuclein protein comprises a nitration of Y39 and Y136.

36. The method of any one of claims 27-35, wherein an increase is a statistically significant increase, wherein the statistically significant increase is an increase is at least a 10%, 25%, 50%, 100%, 2-fold, or 3-fold increase in the concentration of the at least one nitrated alpha-synuclein protein in the CSF relative to the reference level.

37. The method of any one of claims 27-36, wherein the expression level of the nitrated alpha-synuclein protein is calculated as the concentration of the nitrated alpha-synuclein protein in the CSF sample divided by the concentration of total alpha-synuclein in the CSF sample.

38. The method of any one of claims 27-37, wherein the reference expression level is calculated as the concentration of the nitrated alpha-synuclein protein in a reference sample divided by the concentration of total alpha-synuclein in the reference sample.

39. The method of claim 38, wherein the reference sample is a CSF sample from a normal subject that does not have a synucleinopathy.

40. The method of any one of claims 19-39, wherein the synucleinopathy is PD or a subtype of the disease thereof, DLB, or MSA.

41. The method of claim 40, wherein the subtype of the disease thereof is a prodromal stage of PD.

42. The method of any one of claims 27-41, wherein, after administering, the method further comprises determining the protein expression level of a nitrated alpha-synuclein protein comprising a nitration of Y39, Y125, Y133, and/or Y136 in an alpha-synuclein protein in a CSF sample obtained from the subject, wherein a decrease in the expression level of the nitrated alpha-synuclein protein relative to the expression level before administration identifies the subject as having responded to treatment.

43. The method of any one of claims 27-41, wherein, after administering, the method further comprises determining the protein expression level of a nitrated alpha-synuclein protein comprising a nitration of Y39, Y125, Y133, and/or Y136 in an alpha-synuclein protein in a CSF sample obtained from the subject, wherein an increase in the expression level of the nitrated alpha-synuclein protein relative to the expression level before administration identifies the subject as in need of further treatment.

44. A kit for diagnosing a synucleinopathy in a subject, the kit comprising:
(a) probes or antibodies capable of determining the level of one or more of a nitrated alpha-synuclein protein in a CSF sample from the subject; and optionally
(b) instructions for use of the probes or antibodies to determine the level of one or more nitrated alpha-synuclein protein in the CSF sample from the subject, wherein an increase in the level of one or more of the nitrated alpha-synuclein protein relative to a respective reference level indicates that the subject is likely to have a synucleinopathy.

45. The kit of claim 44, wherein the one or more nitrated alpha-synuclein protein is nY39 and nY136.

46. The kit of claim 45, wherein the one or more nitrated alpha-synuclein protein is nY39.

47. The kit of claim 45, wherein the one or more nitrated alpha-synuclein protein is nY136.

48. The kit of any one of claims 44-47, wherein the synucleinopathy is PD or a subtype of the disease thereof, DLB, or MSA.

49. The kit of claim 48, wherein the subtype of the disease thereof is a prodromal stage of PD.
